(19) United States
(12) Patent Application Publication
(10) Pub. No.: US 2026/0174662 A1
(43) Pub. Date: Jun. 25, 2026

(54) COSMETIC COMPOSITION FOR TRICHOLOGICAL ENZYMATIC PEEL FOR THE SCALP AND METHOD OF OBTAINING THEREOF

(71) Applicant: AFLOFARM FARMACJA POLSKA SP Z OO, Pabianice (PL)

(72) Inventors: Hanna WAHL, Pabianice (PL); Marek DABROWA, Lodz (PL); Dagmara CHAJDAS-KOWALCZYK, Poddebice (PL); Marcin OCHOCKI, Lodz (PL); Sylwia DLUZNIK, Ksawerow (PL); Malgorzata DASZKIEWICZ, Lodz (PL); Jaroslaw PASINSKI, Lodz (PL)

(21) Appl. No.: 18/706,953

(22) PCT Filed: Nov. 14, 2022
(86) PCT No.: PCT/PL2022/050079

§ 371 Date: May 2, 2024

(30) Foreign Application Priority Data
Nov. 12, 2021 (PL) P.439492

(51) Int. Cl. A61K 8/66; A61Q 5/02; A61Q 7/00

(52) U.S. Cl. CPC A61K 8/66; A61Q 5/02; A61Q 7/00; A61K 2800/28; A61K 2800/5922

(57) ABSTRACT

The invention relates to a cosmetic composition of a trichological enzymatic peel of the scalp and a method of obtaining thereof.

The invention relates to a cosmetic composition for trichological peel and a method of obtaining thereof. The invention has applications in the cosmetic industry.

International application WO2019237168A1 describes a cosmetic composition for use on hair for smoothing. The composition contains a proteolytic enzyme (cysteine proteinase), such as, for example: papain, bromelain, ficin, cardosine or mixtures thereof; a chelating agent, hydrolyzed protein and a hair dye in salt form. A hair conditioner composition for improving hair shine and colour is known from German patent application DE19709334C1. The conditioner composition is two-component and contains a disulfide bridge degrading agent Pronase E (component A) and a proteolytically active enzyme alkalase (component B), which are stored separately in one package until used. U.S. Pat. No. 5,874,463A skin peeling composition containing alpha and beta hydroxy or keto acids for the topical treatment of hyperpigmentation, chloasma, dyschromia, fine wrinkles, photodamage and skin aging. In addition to the aforementioned acids, the composition also contains kojic acid or its derivatives in a dermatologically acceptable carrier, such as an ethanol/water mixture. A composition containing a proteolytic enzyme, papain, adapted to promote the restoration of primary skin trophism, primary pigmentation and, in particular, to improve the viability of hair follicles, even in the area of scars, is known from European patent application EP1945200A1. The composition contains a mixture of amino acids, biotin, pantothenic acid, vitamin C, retinol, papain, hyaluronic acid and glucose. The composition is designed to treat alopecia, primary hair pigmentation and restore skin trophism. Although a composition for moisturizing and exfoliating the skin can be provided from the state of the art, an analogous composition for use on the scalp and/or hair in humans is not known. Also known from the state of the art is a composition containing enzymes nourishing the skin. Also known is a two-component and composition for use on hair to improve its appearance, where the individual components must be applied in the correct order. Nevertheless, peeling is not the only scalp problem. An additional problem also relates to excessive sebum production, i.e. greasy scalp and hair. Thus, there is still a need for a cosmetic agent for the care of the scalp and/or hair in humans that is convenient to use, without the need to follow the order of application, cleansing the scalp and/or hair, simultaneously preventing the formation of dandruff, moisturizing the skin, stimulating hair growth, as well as regulating the sebaceous glands, thus preventing greasy scalp and/or hair, in the formulation of which no additional preservatives are used. Unexpectedly, the problems presented have been solved by the presented invention.

The first subject of the invention is a cosmetic composition for trichological peel characterized in that it contains from 5.0% to 40.0% of urea (% by weight, number of grams in 100 grams of product), 2% by weight of lactic acid (88% aqueous solution), 3% by weight of salicylic acid, from 1.0% to 1.5% by weight of Saw Palmetto Extract GNBN, from 1.0% to 2.0% by weight of fruit vinegar mixture, preferably Bio Acetum Frutas Acidas, 7% by weight of propylene glycol, from 7.0% to 10.0% by weight of sodium lactate (60% aqueous solution), 0.8% by weight of Prebiulin C90, 2% by weight of Cocamidopropyl Betaine, 0.5% by weight of fragrance composition, 0.1% by weight of bromelain and water to 100% by weight. Bio Acetum Fructas Acidas in its composition preferably contains Vinegar, Pyrus malus fruit extract, Aqua, fructose, glucose, gluconic acid, gluconolactone, Caesalpinia spinosa gum, Citrus limon fruit extract, Tamarindus indica fruit extract. The fragrance composition is preferably Fragrance Composition: Fresh Tea and Cedarwood MOD AR666199. Equally preferably, the composition of the invention contains menthol in an amount of 0.2% by weight.

The second subject of the invention is a method for obtaining the composition defined in the first subject of the invention, characterized in that it includes:
a) preparing solution I by adding salicylic acid to propylene glycol, heating to 40÷45° C. and stirring until dissolved, then adding menthol and stirring again for not less than 10 minutes, until dissolved;
b) preparing solution II by adding Fresh Tea and Cedarwood MOD AR666199 fragrance composition to Cocamidopropyl Betaine and mixing until homogeneous, not less than 5 minutes, avoiding foaming;
c) adding urea to purified water at 55÷60° C., mixing until dissolved. Maintaining the temperature of 40-45° C., introducing sodium lactate (60% aqueous solution) and then stirring a minimum of 10 minutes, continuously maintaining the temperature.
d) adding and stirring solution I to the solution from step c) at the final temperature and continuously maintained;
e) adding lactic acid (88% aqueous solution), Saw Palmetto Extract GNBN, Bio Acetum Frutas Acidas stirring and starting to cool down until reaching 25÷30° C.;
f) adding bromelain, mixing and homogenizing for 10 minutes, then introducing Prebiulin C90, mixing and homogenizing for 30-35 minutes until homogeneous;
e) then introducing solution II and mixing.

The presented invention provided a cosmetic agent for the care of the scalp and/or hair in humans and a method of obtaining thereof, which is convenient to use, without the need to follow the order of application, cleansing the scalp and/or hair, simultaneously preventing the formation of dandruff, moisturizing the skin, stimulating hair growth, as well as regulating the sebaceous glands, thus preventing greasy scalp and/or hair, in the formulation of which no additional preservatives for the composition are used, as well as a method of obtaining thereof.

### EXAMPLE 1

### Development of the Formulation of a Trichological Enzymatic Peel for the Scalp

During the product formulation studies, a number of technological trials were carried out in order to develop an optimal product formulation that provides a unique effect of the composition. The preliminary composition of the product is shown in Table 1.1

TABLE 1.1
Initial product composition

| Starting material | P1 [g] |
|---|---|
| Urea | 5 |
| Lactic acid (88% aqueous solution) | 2 |
| Salicylic acid | 3 |
| Saw Palmetto Extract GNBN (Serenoa Serrulata Fruit | 1 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 2 |
| Propylene glycol | 7 |
| Sodium lactate (60% aqueous solution) | 7 |
| Prebiulin C90* | 0.8 |
| Cocamidopropyl Betaine | 2 |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation Description:

### Preparation of Solution I

Add salicylic acid to propylene glycol, heat to 40÷45° C. and stir until dissolved.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous. Add Cocamidopropyl Betaine, stir.

The product prepared according to the P1 formulation was characterized by too weak scalp cleanliness effect and too weak fragrance due to the ingredients used. Therefore, another technological trial P2 was prepared with the composition disclosed in Table 1.2.

TABLE 1.2
P2 product composition

| Starting material | P2 [g] |
|---|---|
| Urea | 40 |
| Lactic acid (88% aqueous solution) | 2 |
| Salicylic acid | 3 |
| Saw Palmetto Extract GNBN (Serenoa Serrulata Fruit | 1 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 1 |
| Propylene glycol | 7 |
| Sodium lactate (60% aqueous solution) | 10 |
| Prebiulin C90* | 0.8 |
| Cocamidopropyl Betaine | 2 |
| Fragrance composition: Fresh Tea and Cedarwood MOD | 0.15 |
| AR666199 | |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation of the Product:

### Preparation of Solution I

Add salicylic acid to propylene glycol, heat to 40÷45° C. and stir until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous.

Add solution II and mix.

The product prepared according to the P2 formulation was characterized by poor hair smoothing and still too weak fragrance despite the addition of fragrance composition. The next stage of work was the preparation of P3 trial, the composition of which is disclosed in Table 1.3.

TABLE 1.3
P3 product composition

| Starting material | P3 [g] |
|---|---|
| Urea | 35 |
| Lactic acid (88% aqueous solution) | 2 |
| Salicylic acid | 3 |
| Saw Palmetto Extract GNBN (Serenoa Serrulata Fruit | 1 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 2 |
| Propylene glycol | 7 |
| Sodium lactate (60% aqueous solution) | 10 |
| Prebiulin C90* | 0.8 |
| Cocamidopropyl Betaine | 2 |
| Fragrance composition: Fresh Tea and Cedarwood MOD | 0.3 |
| AR666199 | |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation of the Product:

### Preparation of Solution I

Add salicylic acid to propylene glycol, heat to 40÷45° C. and stir until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous.

Add solution II and mix.

A different ratio of urea to Bio Acetum Frutas Acidas and a higher concentration of the fragrance composition was used in the product prepared according to the P3 formulation. Unexpectedly, it turned out that the above-determined ratio of urea to Bio Acetum Frutas Acidas gave satisfactory application properties in terms of the achieved effect, except that the smell of the product was still too weak. The next stage of work involved the preparation of a technological trial according to the P4 formulation.

TABLE 1.4
P4 product composition

| Starting material | P4 [g] |
|---|---|
| Urea | 40 |
| Lactic acid (88% aqueous solution) | 2 |
| Salicylic acid | 3 |
| Saw Palmetto Extract GNBN (Serenoa Serrulata Fruit | 1 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 2 |
| Propylene glycol | 7 |
| Sodium lactate (60% aqueous solution) | 10 |
| Prebiulin C90* | 0.8 |
| Cocamidopropyl Betaine | 2 |
| Fragrance composition: Fresh Tea and Cedarwood MOD | 0.5 |
| AR666199 | |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation of the Product:

### Preparation of Solution I

Add salicylic acid to propylene glycol, heat to 40÷45° C. and stir until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous.

Add solution II and mix.

The product prepared according to the P4 formulation was unexpectedly still characterized by a fragrance that was too weak despite a significant increase in the amount of the odour-correcting substance.

Preparation of the product of the invention with P5 formulation, shown in Table 1.5.

TABLE 1.5
P5 product composition

| Starting material | P5 [g] |
|---|---|
| Urea | 35 |
| Lactic acid (88% aqueous solution) | 2 |
| Salicylic acid | 3 |
| Saw Palmetto Extract GNBN (Serenoa Serrulata Fruit | 1.5 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 2 |
| Propylene glycol | 7 |
| Sodium lactate (60% aqueous solution) | 10 |
| Prebiulin C90* | 0.8 |
| Cocamidopropyl Betaine | 2 |
| Fragrance composition: Fresh Tea and Cedarwood MOD | 0.5 |
| AR666199 | |
| Menthol | 0.2 |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation of Solution I

Add salicylic acid to propylene glycol, heat to 40÷45° C. and stir until dissolved, then add menthol and stir again for no less than 10 minutes, until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous.

Add solution II and mix.

Unexpectedly, it was found that the addition of menthol to the P5 formulation improved the fragrant properties of the product.

The product according to the P5 formulation was characterized by acceptable application qualities and became the starting point for further studies: the selection of a preservative additive and the selection of an enzyme for the optimal effect.

### EXAMPLE 2

Determining how to preserve the product.

It is routine for cosmetic products to contain additional preservatives in the formulation. During the research work on this product, formulations with added preservative were also prepared: P6 trial with the formulation shown in Table 3.6.

TABLE 2.6
Composition of the product including a preservative.

| Starting material | P6 [g] |
|---|---|
| Urea | 35 |
| Lactic acid (88% aqueous solution) | 2 |
| Salicylic acid | 3 |
| Saw Palmetto Extract GNBN (Serenoa Serrulata Fruit | 1.5 |
| Extract 0.5%-2.5%; Glycerin 40%-60%; Aqua 40%-60%; | |
| Sodium Benzoate 0.2%-0.3%; Potassium Sorbate 0.2%-0.3%) | |
| Bio Acetum Frutas Acidas | 2 |
| Propylene glycol | 7 |
| Sodium lactate (60% aqueous solution) | 10 |
| Prebiulin C90* | 0.8 |
| Cocamidopropyl Betaine | 2 |
| Fragrance composition: Fresh Tea and Cedarwood MOD | 0.5 |
| AR666199 | |
| Menthol | 0.2 |
| Euxyl K712 | 0.2 |
| Purified water | Ad 100 g |

*Prebiulin C90 composition: Cellulose Gum 50%-70%, Xanthan Gum 5%-10%, Inulin 5%-10%, Cellulose <5%, Fructose <5%, Glucose <5%

### Preparation of the Product:

### Preparation of Solution I

Add salicylic acid to propylene glycol, heat to 40÷45° C. and stir until dissolved, then add menthol and stir again for no less than 10 minutes, until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas while stirring, start cooling down to 25÷30° C. Then add Prebiulin C90, stir and homogenize for 30-35 minutes until homogeneous.

Add solution II and mix.

The above P6 formulation was tested for preservation efficacy. An experiment was performed to confirm the efficacy and evaluate the preservation of the cosmetic product. The experiment was performed according to the PN-EN ISO 11930:2012 international standard and consisted of a single introduction of test strains of Escherichia coli, Staphylococcus aureus, Pseudomonas aeruginosa, Candida albicans and Aspergillus brasiliensis to simulate secondary infection of the product by a consumer. Inoculated samples were incubated at 22° C. in a darkened area. For each strain, the amount of colony-forming microorganisms was tested at 7, 14 and 28 days after inoculation. The test showed that the amount of microorganisms found (in cfu/g) was below the limit of quantification for each of the tested strains, and the reduction values on a logarithmic scale exceeded the minimum values necessary to meet Criterion A specified in the above-mentioned standard. Testing of the cosmetic sample proved that it meets Criterion A specified in PN-EN ISO 11930:2012, i.e. it is protected against the growth of microorganisms that may be a hazard for the user.

TABLE 4
The amount of microorganisms Nx in
the test sample at t₇, t₁₄ and t₂₈.

Number of microorganisms
(cfu/1 g) in time

| Test strain | N₇ | N₁₄ | N₂₈ |
|---|---|---|---|
| Escherichia coli | <300 | <300 | <300 |
| Staphylococcus aureus | <300 | <300 | <300 |
| Pseudomonas aeruginosa | <300 | <300 | <300 |
| Candida albicans | <300 | <300 | <300 |
| Aspergillus brasiliensis | — | <150 | <150 |

| | Value of logarithmic | | |
|---|---|---|---|
| | reduction Rx in time | | |
| Test strain | R₇ | R₁₄ | R₂₈ |
| Escherichia coli | >3.4 | >3.4 | >3.4 |
| Staphylococcus aureus | >3.3 | >3.3 | >3.3 |
| Pseudomonas aeruginosa | >3.4 | >3.4 | >3.4 |
| Candida albicans | >2.2 | >2.2 | >2.2 |
| Aspergillus brasiliensis | — | >2.5 | >2.5 |

A “<” or “>” sign indicates that the result lies “above” or “below” the limit of quantification.

The same tests were also carried out on the P5 formulation product, which did not contain an additional preservative in its composition. The sample prepared according to P6 passed the preservation test, unexpectedly it turned out that the P5 sample, which did not contain an additional preservative, also passed the preservation test, and in the interest of the users this was the composition chosen for further work. Surprisingly, the result for the P5 sample was concordant with the result for the P6 sample and also meets the Criterion A according to PN-EN ISO 11930:2012, i.e. it is protected against the growth of microorganisms that may be a hazard for the user.

TABLE 4
The amount of microorganisms Nx in
the test sample at t₇, t₁₄ and t₂₈.

Number of microorganisms
(cfu/1 g) in time

| Test strain | N₇ | N₁₄ | N₂₈ |
|---|---|---|---|
| Escherichia coli | <300 | <300 | <300 |
| Staphylococcus aureus | <300 | <300 | <300 |
| Pseudomonas aeruginosa | <300 | <300 | <300 |
| Candida albicans | <300 | <300 | <300 |
| Aspergillus brasiliensis | — | <150 | <150 |

A “<” or “>” sign indicates that the result lies “above” or “below” the limit of quantification, respectively.

TABLE 5
Rx reduction value for each test strain at
t₇, t₁₄ and t₂₈ expressed in logarithmic units.

| | Value of logarithmic | | |
|---|---|---|---|
| | reduction Rx in time | | |
| Test strain | R₇ | R₁₄ | R₂₈ |
| Escherichia coli | >3.4 | >3.4 | >3.4 |
| Staphylococcus aureus | >3.4 | >3.4 | >3.4 |
| Pseudomonas aeruginosa | >3.4 | >3.4 | >3.4 |
| Candida albicans | >2.3 | >2.3 | >2.3 |
| Aspergillus brasiliensis | — | >2.5 | >2.5 |

A “<” or “>” sign indicates that the result lies “above” or “below” the limit of quantification, respectively.

### EXAMPLE 3

The selection of an enzyme compatible with the pre-developed formulation was carried out in trials P7, P8, P9, with the compositions disclosed in Table 3.1.

TABLE 3.1
Selection of enzyme for initial product formulation

| Starting material | P7 | P8 | P9 |
|---|---|---|---|
| Urea | 35 | 35 | 35 |
| Lactic acid (88% aqueous solution) | 2 | 2 | 2 |
| Salicylic acid | 3 | 3 | 3 |
| Saw Palmetto Extract GNBN (Serenoa | 1.5 | 1.5 | 1.5 |
| Serrulata Fruit Extract 0.5%-2.5%; | | | |
| Glycerin 40%-60%; Aqua 40%-60%; | | | |
| Sodium Benzoate 0.2%-0.3%; | | | |
| Potassium Sorbate 0.2%-0.3%) | | | |
| Bio Acetum Frutas Acidas | 2 | 2 | 2 |
| Propylene glycol | 7 | 7 | 7 |
| Sodium lactate (60% aqueous solution) | 10 | 10 | 10 |
| Prebiulin C90* | 0.8 | 0.8 | 0.8 |
| Cocamidopropyl Betaine | 2 | 2 | 2 |
| Fragrance composition: Fresh Tea | 0.5 | 0.5 | 0.5 |
| and Cedarwood MOD AR666199 | | | |
| Menthol | 0.2 | 0.2 | 0.2 |
| Bromelain (Bromelain 95%, | 0.1 | — | — |
| Maltodextrin 5%) | | | |
| Papain | — | 0.1 | |
| Lactobacillus/Pumpkin Fruit | — | — | 0.1 |
| Ferment Filtrate | | | |
| Purified water | Ad | Ad | Ad |
| | 100 g | 100 g | 100 g |

*Prebiulin C90 composition: Cellulose Gum 50%-70%, Xanthan Gum 5%-10%, Inulin 5%-10%, Cellulose <5%, Fructose <5%, Glucose <5%

### Product Preparation:

### Preparation of Solution I

Add salicylic acid to propylene glycol, heat to 40÷45° C. and stir until dissolved, then add menthol and stir again for no less than 10 minutes, until dissolved.

### Preparation of Solution II

Add Fresh Tea and Cedarwood MOD AR666199 fragrance composition to Cocamidopropyl Betaine and mix until homogeneous, not less than 5 minutes, avoiding foaming.

### Preparation of the Product

Heat purified water to 55÷60° C. and add urea, stir until dissolved. Maintaining the temperature at 40-45° C., add sodium lactate (60% aqueous solution) and then stir for a minimum of 10 minutes, maintaining the temperature continuously.

Maintaining the temperature, add solution I and mix.

Add lactic acid (88% aqueous solution), Saw Palmetto Extract GNBN, Bio Acetum Frutas Acidas, stir and start cooling down to 25÷30° C. Add bromelain in P7, papain in P8, Lactobacillus/Pumpkin Fruit Ferment Filtrate in P9, stir and homogenize 10 minutes. Then add Prebiulin C90, stir and homogenize for 30-35 minutes until homogeneous.

Add solution II and mix.

Among the P7-P9 trials prepared according to the above description, acceptable technological properties of the product were obtained only for the P7 trial. In addition, the composition of P7 unexpectedly was the only one that met the application assumptions, and therefore was considered the final composition, which was submitted for in-use tests of the cosmetic product.

Below are sources of substances for which the INCI name was not given in the description:

| | |
|---|---|
| Prebiulin C90 | https://www.amitahc.com/pcare_ingredient/ |
| | prebiulin-c90/ |
| Fresh Tea and Cedarwood | http://www.alfasagittarius.eu/ |
| MOD AR666199 fragrance | |
| composition | |
| Euxyl K712 | https://www.brenntag.com/pl-pl/index.html |
| Saw Palmetto Extract | https://www.weareprovital.com/pl/ |
| GNBN | caremotives/vitacares/ekstrakt-z-sabal |
| Bio Acetum Frutas Acidas | https://www.cqmasso.com/fcr/ |

### EXAMPLE 4

### In-Use Tests of the Cosmetic Product

The product of the invention, prepared as described in Example 3 with the P7 formulation, was subjected to in-use tests, the purpose of which was, among other, to evaluate the skin tolerance of the applied product among volunteers.

The in-use test was performed with 30 healthy volunteers under home conditions during 3 weeks of regular, repeated application. The study showed good skin tolerance of the product during the application period when applied as intended and following the instructions of use. There were no symptoms indicating intolerance or hypersensitivity to any of the product's ingredients. As expected, the volunteers overwhelmingly confirmed the product's intended properties in terms of scalp cleansing, removal of dead skin and skin refreshment, hair lifting effect, the effect persistence and scalp comfort, scalp care evaluation, effect on greasy scalp and itching, scalp appearance, stimulation of hair growth and reduction of hair loss, improvement of follicle condition, and scalp hydration.

Unexpectedly, most of the respondents found that the applied product gave better and faster results than the scalp peels used so far. Moreover, unexpectedly, the product showed a normalizing effect on sebum secretion and reduced greasy hair, restored balance to the scalp and had an anti-dandruff effect. It was also unexpectedly found that the product strengthens hair and restores hair shine.

Results of the survey conducted:
Does the product thoroughly cleanse the scalp surface after the very first application? (97% positive responses)
Does the product effectively remove dead skin present on the scalp after the very first application? (93% positive responses)
Does the product provide a visible effect of cleansing and refreshing the scalp after a single application? (97% positive responses)
Does the product provide a spectacular effect after a single application, leaving the hair lifted at the roots? (80% of positive responses)
Does the product leave a long-lasting fresh scalp effect? (93% positive responses)
Is the product indispensable in scalp care? (77% of positive answers)
Does the product give better results than the scalp peels you have used so far? (If you have not used this type of product, please skip the question) (79% of positive answers)
Does the product give faster results than the scalp peels you have used so far? (If you have not used this type of product, please skip the question) (79% positive responses)
Does the product intensively dissolve dead skin extending the freshness of the scalp and hair? (87% of positive answers)
Does the product not irritate the scalp? (93% of positive answers)
Does the product effectively restore comfort to the scalp? (93% of positive responses)
Does the product effectively restore healthy appearance of the scalp by removing impurities and dead skin? (93% positive responses)
Does the product normalize sebum secretion and reduce greasy hair, leaving it fresh for longer? (83% positive responses)
Does the product reduce oily scalp? (77% of positive responses)
Does the use of the product provide hair lifting at the roots? (70% of positive responses)
Does the product restore balance to the scalp, reducing hair loss by strengthening the hair? (77% of positive responses)
Does the use of the product prevent dandruff formation? (87% of positive responses)
Is the product designed for a demanding scalp that is greasy and itchy? (83% of positive responses)
Is the product effective in restoring hair shine? (73% of positive answers)
Is the product effective in strengthening the hair? (87% of positive responses)
Is the product effective in stimulating new hair growth? (77% of positive responses)

Is the product effective in stimulating regrowth and accelerating hair growth? (70% of positive responses)
Is the product effective in reducing hair loss? (77% of positive responses)
Does the product dramatically improve the condition of hair follicles and the condition of the scalp? (90% of positive responses)
Does regular use of the product have a positive effect on the hair appearance? (93% of positive answers)
Does regular use of the product have a regenerative effect on the scalp, leaving it refreshed, moisturized and free of flaky skin? (93% positive responses)
Is the product ideal for those with a dry scalp characterized by itching? (90% positive responses)
Does the product have an anti-dandruff effect? (80% of positive responses)
Does the use of the product leave the hair fresh for a long time? (83% of positive answers)
Does the use of the product leave the scalp moisturized, so it doesn't itch or flake? (90% of positive responses)
Does the use of the product favourably affect the hair condition and make it shiny and strengthened? (83% positive responses)
After regular use of the product, is the hair strengthened from the root to the entire length, making it resistant to damage and breakage? (87% positive responses)
Does the product provide spectacular results after the first application, leaving the scalp cleansed, refreshed and moisturized? (83% positive responses)
Does the product provide spectacular results after the very first application, leaving the hair fresh and lifted at the roots? (77% positive responses)
Does the product applicator provide easy and quick application? (97% positive responses)
Does the product enhance the effectiveness of other hair care products? (80% of positive responses)
Does the product's packaging provide hassle-free and precise application? (97% of positive responses)

1. A cosmetic composition for trichological peel, characterized in that it contains 5.0% to 40.0% of urea (% by weight, number of grams in 100 grams of product), 2% by weight of lactic acid (88% aqueous solution), 3% by weight of salicylic acid, from 1.0% to 1.5% by weight of Saw Palmetto Extract GNBN, from 1.0% to 2.0% by weight of fruit vinegar mixture, preferably Bio Acetum Frutas Acidas, 7% by weight of propylene glycol, from 7.0% to 10.0% by weight of sodium lactate (60% aqueous solution), 0.8% by weight of Prebiulin C90, 2% by weight of Cocamidopropyl Betaine, 0.5% by weight of fragrance composition, 0.1% by weight of bromelain and water to 100% by weight of solution.

2. The composition of claim 1, characterized in that it contains menthol in an amount of 0.2% by weight.

3. A method for obtaining the composition of claim 2 comprising:
a) preparing solution I by adding salicylic acid to propylene glycol, heating to 40 to 45° C. and stirring until dissolved, then adding menthol and stirring again for not less than 10 minutes, until dissolved;
b) preparing solution II by adding Fresh Tea and Cedarwood MOD AR666199 fragrance composition to Cocamidopropyl Betaine and mixing until homogeneous, not less than 5 minutes, avoiding foaming;
c) adding urea to purified water at 55 to 60° C., mixing until dissolved. Maintaining the temperature of 40 to 45° C., introducing sodium lactate (60% aqueous solution) and then stirring a minimum of 10 minutes, continuously maintaining the temperature
d) adding and stirring solution I to the solution from step c) at the final temperature and continuously maintained;
e) adding lactic acid (88% aqueous solution), Saw Palmetto Extract GNBN, Bio Acetum Frutas Acidas stirring and starting to cool down until reaching 25 to 30° C.;
f) adding bromelain, mixing and homogenizing for 10 minutes, then introducing Prebiulin C90, mixing and homogenizing for 30-35 minutes until homogeneous;
e) then introducing solution II and mixing.

4. A method for obtaining the composition of claim 1 comprising:
a) preparing solution I by adding salicylic acid to propylene glycol, heating to 40 to 45° C. and stirring until dissolved;
b) preparing solution II by adding Fresh Tea and Cedarwood MOD AR666199 fragrance composition to Cocamidopropyl Betaine and mixing until homogeneous, not less than 5 minutes, avoiding foaming;
c) adding urea to purified water at 55 to 60° C., mixing until dissolved. Maintaining the temperature of 40 to 45° C., introducing sodium lactate (60% aqueous solution) and then stirring a minimum of 10 minutes, continuously maintaining the temperature.
d) adding and stirring solution I to the solution from step c) at the final temperature and continuously maintained;
e) adding lactic acid (88% aqueous solution), Saw Palmetto Extract GNBN, Bio Acetum Frutas Acidas stirring and starting to cool down until reaching 25 to 30° C.;
f) adding bromelain, mixing and homogenizing for 10 minutes, then introducing Prebiulin C90, mixing and homogenizing for 30-35 minutes until homogeneous;
e) then introducing solution II and mixing.
